(19) United States
(12) Patent Application Publication
(10) Pub. No.: US 2007/0292451 A1
(43) Pub. Date: Dec. 20, 2007

(54) Medical herb composition for promoting the growth of hair A mammal and the manufacturing method thereof

(75) Inventors: Shuenn-Jyi Sheu, Taipei City (TW); Hsiao-Yin Yeh, Taipei City (TW); Chung-Lin Yu, Taoyuan City (TW); Wu-Che Wen, Danshuei Township (TW); Ching-Che Lin, Taipei City (TW); Chen-Ying Chiang, Taipei City (TW)

Correspondence Address: BACON & THOMAS, PLLC, 625 SLATERS LANE, FOURTH FLOOR, ALEXANDRIA, VA, 22314, US

(73) Assignee: Brion Research Institute of Taiwan, Taipei (TW)

(21) Appl. No.: 11/806,540

(22) Filed: Jun. 1, 2007

(30) Foreign Application Priority Data
Jun. 16, 2006 (TW) 095121638

(51) Int. Cl. A61K 36/489; A61K 36/00; A61K 36/06; A61K 36/232; A61K 36/535; A61K 36/804; A61Q 19/00; A61Q 7/00; A61K 36/9068; A61K 36/539; A61K 36/258; A61K 36/48; A61K 36/484

(52) U.S. Cl. 424/195.15; 424/728; 424/745; 424/773; 424/775; 424/776; 424/777; 424/779

(57) ABSTRACT

A medical herb composition for promoting the growth of hair of a mammal is disclosed. The medical herb composition comprises: an extract of Sophorae Radix; an extract of at least one vital-energy-invigorating plant; and an extract of at least one blood-nourishing plant. Moreover, the weight percentage of the extract of Sophorae Radix is 2-38, the weight percentage of the extract of the vital-energy-invigorating plant is 2-60, and the weight percentage of the extract of the blood-nourishing plant is 2-60. In addition, the medical herb composition can optionally further comprise an extract of at least one skin-pathogen-expelling plant, an extract of at least one diuresis-inducing plant, an extract of at least one heat-clearing plant, an extract of at least one spleen-invigorating plant, or/and an extract of at least one dampness-removing plant. The disclosed medical herb composition can also be used to treat skin disorders of a mammal.

### BACKGROUND OF THE INVENTION

1. Field of the Invention

The present invention relates to a medical herb composition, particularly to a medical herb composition for promoting the growth of hair.

2. Description of Related Art

Sophorae Radix has certain therapeutic effects on skin disorders, and its use as a promoter for the growth of hair is known in the art, as disclosed in JP2002205922, which evaluates the promotion effect of an extract of genus Sophra on the growth of hair follicles by in vitro assays.

JP4202117 discloses the possibility of promoting the growth of hair follicles by an extract of genus Sophra or the like. However, the inventions set forth only show in vitro assays of an extract of genus Sophra or the like, and no examples regarding practices on higher animals or humans to demonstrate its efficacy. Generally, there is still a long way to go from in vitro experiments to practicing on humans. In addition, there is no prior art disclosing the clear efficacy on promoting the growth of hairs of higher animals and humans by oral administration of an extract of Sophorae Radix. One of the inventors of the present invention, Dr. Chung-Lin Yu treated animal skin disorders with the composition comprising an extract of Sophorae Radix as the main ingredient. Though the composition could significantly improve the conditions of itchness, erythema, and exormia, the effect on promoting the growth of hair was not observed. (Chung-Lin Yu, Comparative Studies on Treating Canine Skin Disorders with Modern and Herbal Medicines, master's thesis, 1995, Taipei). The study illustrates that the extract of Sophra Radix, that was proven to promote the growth of hair follicles in vitro and also have therapeutic efficacy on skin disorders, cannot perform promotion effect on the growth of hair follicles when orally administered to higher animals. The vital-energy-invigorating plants (such as Ginseng Radix and Astragali Radix) and the blood-nourishing plants (such as Angelicae Radix and Rehmanniae Preparata Radix) often used as herbal food supplements have pharmacologically proven effects on anti-secession, strengthening, facilitating learning and memory, lowering blood sugar, helping digestion, absorption, and metabolism, improving immunity, anti-tumor, anti-clotting, anti-allergy, anti-heart-myoischemia, stimulating haemopoiesis, prevention of cardiovascular diseases, sedation, pain-relief, and kidney protection. However, no study or publication to date proves that those plants and their extracts can promote the growth of hair.

The present invention utilizes an extract of Sophorae Radix in conjunction with certain herbs to produce a novel composition, and the assays conducted on the basis of oral administration to higher animals and humans are far more straightforward and convincing than in vitro experiments. By observing multiple sets of experiments on animals and humans, it is found that the effect on promoting the growth of hair is remarkable when the composition of the present invention is administrated, and the growth rate outperforms current records in related literatures. The usage of the composition of the present invention is easier than the commercially available tonics that are administered by daubing, and the composition of the present invention does not cause any side-effect observed in the usage of the commercially available tonics. In addition, the clear-cut efficacy on relieving animal as well as human skin disorders of the composition is demonstrated.

These and other objects of the present invention will be apparent from the detailed description of the invention provided hereinafter.

### SUMMARY OF THE INVENTION

The present invention discloses a medical herb composition, comprising: an extract of Sophorae Radix; an extract of at least one vital-energy-invigorating plant; and an extract of at least one blood-nourishing plant, wherein the weight percentage of the extract of Sophorae Radix is 2˜38, the weight percentage of the extract of the vital-energy-invigorating plant is 2˜60, and the weight percentage of the extract of the blood-nourishing plant is 2˜60.

The medical herb composition can further comprise: an extract of at least one skin-pathogen-expelling plant, an extract of at least one diuresis-inducing plant, an extract of at least one heat-clearing plant, or/and an extract of at least one-invigorating and dampness-removing plant.

The extracts can be extracted by water or alcohol.

In the medical herb composition of the present invention, the vital-energy-invigorating plant can be any conventional one. Preferably, the vital-energy-invigorating plant is selected from the group consisting of: Ginseng Radix, Astragali Radix, Batatatis Rhizoma, Zizyphi Fructus, and Tremella.

The blood-nourishing plant in the medical herb composition of the present invention can be any conventional one. Preferably, the blood-nourishing plant is selected from the group consisting of: Angelicae Radix, Rehmanniae Preparata Radix, Longanae Arillus, and Lycii Fructus.

In addition, the present invention also discloses a method for preparing a medical herb composition for promoting the growth of hair of a mammal, comprising the following steps: (a) providing an extract of Sophorae Radix, an extract of at least one vital-energy-invigorating plant, and an extract of at least one blood-nourishing plant by individual extraction and drying; and (b) mixing the dried extracts to form a mixture, wherein the vital-energy-invigorating plant is selected from the group consisting of: Ginseng Radix, Astragali Radix, Batatatis Rhizoma, Zizyphi Fructus, and Tremella; and the blood-nourishing plant is selected from the group consisting of: Angelicae Radix, Rehmanniae Preparata Radix, Longanae Arillus, and Lycii Fructus. In the medical herb composition of the present invention, the weight percentage of the extract of Sophorae Radix is 2˜38, the weight percentage of the extract of vital-energy-invigorating plant is 2˜60, and the weight percentage of the extract of blood-nourishing plant is 2˜60.

In the method for preparing a medical herb composition for promoting the growth of hair of a mammal, the extract of at least one vital-energy-invigorating plant is preferably an extract of Ginseng Radix or/and an extract of Astragali Radix, and the extract of at least one blood-nourishing plant is preferably an extract of Angelicae Radix.

Step (a) of the present invention can further comprise: providing 0.1 wt %-50 wt % of an extract of at least one skin-pathogen-expelling plant, preferably 5 wt %˜40 wt %; 0.1 wt %-50 wt % of an extract of at least one diuresis-inducing plant, preferably 5 wt %˜40 wt %; 0.1 wt %-70 wt % of an extract of at least one heat-clearing plant, preferably 5%˜60; and/or 0.1 wt %-40 wt % of an extract of at least one spleen-invigorating plant dampness-removing plant, preferably 2%˜30%.

In the medical herb composition of the present invention, the optionally used skin-pathogen-expelling plant can be any conventional one. Preferably, the skin-pathogen-expelling plant is selected from the group consisting of: Ledebouriellae Radix, Notopterygii Rhizoma, Cimicifugae Rhizoma, Allii Fistulosi Bulbus, Asari Herba Cum Radice, Ephedrae Herba, Perillae Folium, and Zingiberis Rhizoma.

In the medical herb composition of the present invention, the optionally used diuresis-inducing plant can be any conventional one. Preferably, the diuresis-inducing plant is selected from the group consisting of: Alismatis Rhizoma, Polyporus, Atractylodis Rhizoma, Akebiae Caulis, Fangchi Radix, and Boehmeriae Radix.

In the medical herb composition of the present invention, the optionally used heat-clearing plant can be any conventional one. Preferably, the heat-clearing plant is selected from the group consisting of: Puerariae Radix, Scutellariae Radix, Anemarrhenae Rhizoma, Glycyrrhizae Radix, Artemisiae Capillaris Herba, Rehmanniae Radix, and Moutan Radicis Cortex.

In the medical herb composition of the present invention, the optionally used spleen-invigorating and dampness-removing plant (aromatic dampness-removing plant) can be any conventional one. Preferably, the spleen-invigorating and dampness-removing plant is selected from the group consisting of: Atractylodis Rhizoma, Eupatorii Herba, Magnolia Cortex, and Pogostemi Herba.

Preferably, the extract of at least one skin-pathogen-expelling plant is an extract of Ledebouriellae Radix, an extract of Notopterygii Rhizoma, or/and an extract of Cimicifugae Rhizoma; the extract of at least one diuresis-inducing plant is an extract of Alismatis Rhizoma, an extract of Polyporus, or/and an extract of Atractylodis Rhizoma; the extract of at least one heat-clearing plant is an extract of Puerariae Radix, an extract of Scutellariae Radix, an extract of Anemarrhenae Rhizoma, an extract of Glycyrrhizae, or/and an extract of Artemisiae Capillaris Herba; the extract of at least one spleen-invigorating and dampness-removing plant is an extract of Atractylodis Rhizoma.

Step (b) of the method for preparing a medical herb composition of the present invention can further comprise: providing an excipient, and mixing the excipient and the mixture to form the medical herb composition. If necessary, other pharmaceutical acceptable carriers can be further added. For example, they can be used in orally administered medical herb composition, and the formulations include: capsules, tablets, emulsifiers, liquid suspensions, dispersants, and solvents. Taking tablets as an illustration, the commonly used carriers are galactose or cornstarch; and lubricants and magnesium stearate are basic additives. When oral capsules are used, galactose and dried corn starch can act as effective diluting agents. Suitable sweetening agents, seasoning agents or pigments can be added if necessary. Preferably, the medical herb composition of the present invention can further comprise an excipient, which can be any conventional one, preferably corn starch.

The extracts in the medical herb composition of the present invention can be prepared by conventional methods for preparing Scientifically Concentrated Traditional Chinese Medicines. Preferably, the extracts are prepared by the method disclosed in the present invention. More preferably, the extracts are provided by water extraction, granulated by spray-drying (or freeze-drying), and then mixed proportionally.

When the medical herb composition containing an extract of Sophorae Radix is employed as an orally administered medicine, it produces the superb inhibiting effect on wet-skin disorder (erythema, eczema etc.). Meanwhile, the medical herb composition of the present invention possesses unexpectedly notable capability in promoting the growth of hair of a mammal. By the experiments administering the medical herb composition to mammals (e.g. humans, dogs, and so on), the remarkable performance of promoting the growth of hair of a mammal is proven.

### BRIEF DESCRIPTION OF THE DRAWINGS

FIG. 1(a)-(c) shows the comparison of photos before and after administration of the medical herb composition of Example 1.

FIG. 2(a)-(d) shows the comparison of photos before and after administration of the medical herb composition of Example 2.

FIG. 3(a)-(b) shows the comparison of photos before and after administration of the medical herb composition of Example 3.

FIGS. 4(a1)-(a2), (b1)-(b2) shows the comparison of photos before and after administration of the medical herb composition of Example 4.

FIG. 5(a)-(b) shows the comparison of photos before and after administration of the medical herb composition in Example 5.

FIGS. 6(a1)-(a3), (b1)-(b3) shows the comparison of photos before and after administration of the medical herb composition of Example 6.

FIG. 7(a)-(b) shows the comparison of photos before and after administration of the medical herb composition in Example 7.

FIG. 8(a)-(b) shows the comparison of photos before and after administration of the medical herb composition in Example 13.

FIG. 9(a)-(b) shows the comparison of photos before and after administration of the medical herb composition in Example 14.

### DETAILED DESCRIPTION OF THE PREFERRED EMBODIMENT

By conventional methods of preparation for Scientifically Concentrated Traditional Chinese Medicines including extraction, separation, concentration, and dryness (as disclosed in Sun-Tien Medical Journal 4 and 5, 2005), the extract powders of each plant were obtained and used in experiments.

The present invention discloses using an extract of Sophorae Radix in conjunction with an extract of at least one vital-energy-invigorating plant (Ginseng Radix/Astragali Radix), and an extract of at least one blood-nourishing plant (Angelicae Radix), and contingently adding an extract of skin-pathogen-expelling plant (Ledebouriellae Radix/Notopterygii Rhizoma/Cimicifugae Rhizoma), an extract of at least one diuresis-inducing plant (Alismatis Rhizoma/Polyporus/Atractylodis Rhizoma), an extract of at least one heat-clearing plant (Puerariae Radix/Scutellariae Radix/Anemarrhenae Rhizoma/Glycyrrhizae Radix/Artemisiae Capillaris. Herba), and/or: an extract of at least one spleen-invigorating and dampness-removing plant (Atractylodis Rhizoma) proportionally to generate the medical herb composition. The components of the five medical herb compositions are illustrated respectively in Table 1-5.

The administered dose is based on body weight. For dogs and cats, the dose was 1-1.5 g/5 kg/day. For humans, the dose was 1.5-2 g/10 Kg/day. The dose for dogs and cats is double that of humans because dogs and cats have higher metabolism rate than humans.

### Example 1

Composition #1 was taken, comprising the components as shown in Table 1.

TABLE 1
Composition # 1

| | Plant | Weight (%) |
|---|---|---|
| | Sophorae Radix | 38.7 |
| skin-pathogen-expelling | Ledebouriellae | 29.0 |
| plant | Radix/Notopterygii | |
| | Rhizoma | |
| diuresis-inducing plant | Alismatis Rhizoma | 25.8 |
| heat-clearing plant | — | — |
| spleen-invigorating plant | — | — |
| dampness-removing | | |
| plant | | |
| vital-energy-invigorating | Ginseng Radix | 3.2 |
| plant | | |
| blood-nourishing plant | Angelicae Radix | 3.2 |

Weight (%) means the weight percentage of the extract of the plant.

The example was practiced on a female Pit Bull Terrier aged 15 years old and weighing 27 Kg. Clinically, the lesions were chronic, and the conditions of lichenification, increasing thickness of skin, and alopecia were observed. Furthermore, erythema, scale, and papule were also identified. The dog was diagnosed to suffer from superficial pyoderma and alopecia.

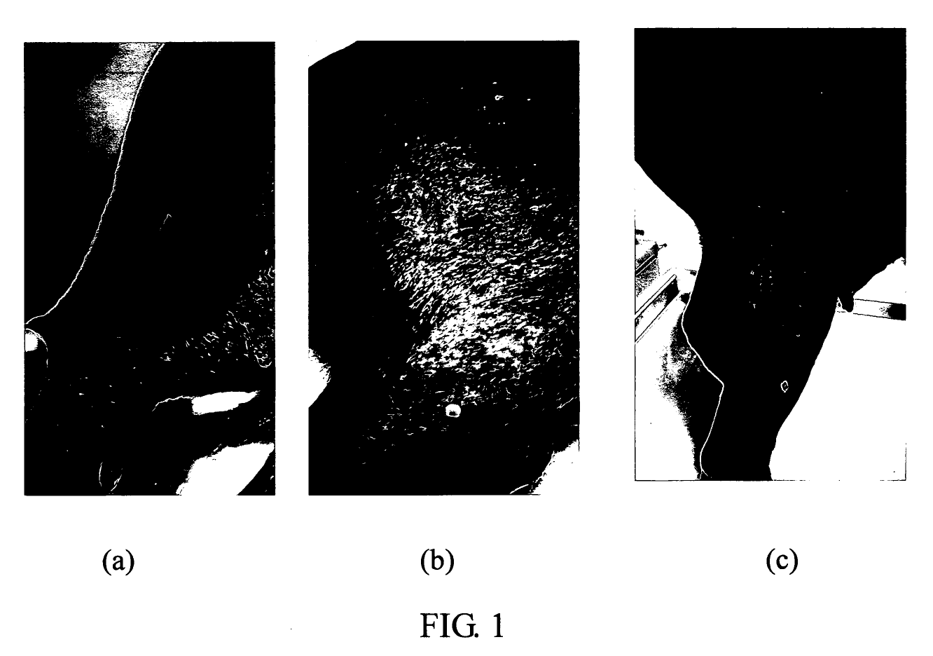

The composition of the present invention was administered after treatment with medical herb itch-relieving agents for a week. The results are shown in FIG. 1. FIG. 1(a) illustrates the subject before treatment, wherein scales and papules were clearly visible; FIG. 1(b) illustrates the subject after administration of medical herb itch-relieving agents for one week, wherein the conditions of scales and papules had been improved; FIG. 1(c) illustrates the subject after administration of the composition of the present invention for two weeks. Obviously, the hair of the dog gradually recovered after administration of the composition.

### Example 2

Composition #2 was taken, comprising the components as shown in Table 2.

TABLE 2
Composition #2

| | Plant | Weight (%) |
|---|---|---|
| | Sophorae Radix | 12.9 |
| skin-pathogen-expelling | Notopterygii | 17.7 |
| plant | Rhizoma/ | |
| | Cimicifugae | |
| | Rhizoma | |
| diuresis-inducing plant | — | — |
| heat-clearing plant | Puerariae Radix, | 6.5 |
| | Scutellariae Radix | |
| spleen-invigorating plant | Atractylodis | 6.5 |
| dampness-removing | Rhizoma | |
| plant | | |
| vital-energy-invigorating | Ginseng Radix | 30.6 |
| plant | | |
| blood-nourishing plant | Angelicae Radix | 25.8 |

Weight (%) means the weight percentage of the extract of the plant.

A 2-year-old female Schnauzer was employed in this example. Clinically, the conditions of ulcers on both inner and outer sides of each ear, and considerable scurf but no pruritus were observed. The aforementioned conditions had been treated but the effect was poor. No parasite was observed by microscopic observation. The dog was diagnosed to suffer from deep pyoderma.

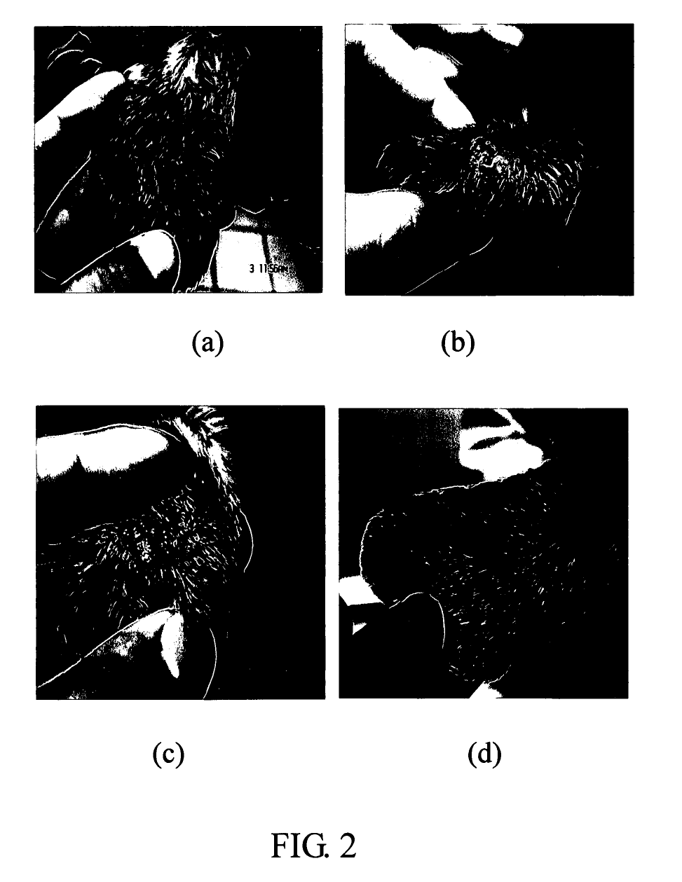

The composition of the present invention was administered for one week after treatment with medical herb itch-relieving agents for two weeks. The results are shown in FIG. 2. FIG. (2a) illustrates the subject before treatment, wherein ulcers were clearly visible. FIG. 2(b) and FIG. 2(c) illustrate the subject after administration of medical herb itch-relieving agents for one week and two weeks, respectively, wherein the condition of ulcers had been improved. FIG. 2(d) illustrates the subject after administration of the composition of the present invention for one week. Obviously, the hair of the dog gradually recovered after administration of the composition.

### Example 3

Composition #3 was taken, comprising the components as shown in Table 3.

TABLE 3
Composition #3

| | Plant | Weight (%) |
|---|---|---|
| | Sophorae Radix | 3.2 |
| skin-pathogen-expelling | Notopterygii | 0.0 |
| plant | Rhizoma/ | |
| | Cimicifugae | |
| | Rhizoma | |
| diuresis-inducing plant | Polyporus | 24.2 |
| heat-clearing plant | Glycyrrhizae | 33.9 |
| | Radix/Artemisiae | |
| | Capillaris Herba | |
| spleen-invigorating plant | Atractylodis | 6.5 |
| dampness-removing | Rhizoma | |
| plant | | |
| vital-energy-invigorating | Ginseng Radix | 9.7 |
| plant | | |
| blood-nourishing plant | Angelicae Radix | 22.6 |

Weight (%) means the weight percentage of the extract of the plant.

A male Siberian Husky was employed in this example. Clinically, the skin disorders of the subject had persisted over one year, and mainly were characterized as itchness and scales. The dog had been treated by coordinately administering antibiotics, steroids and anti-histamine, and aided treatments, but the effect was limited. The dog was diagnosed to suffer from shallow pyoderma.

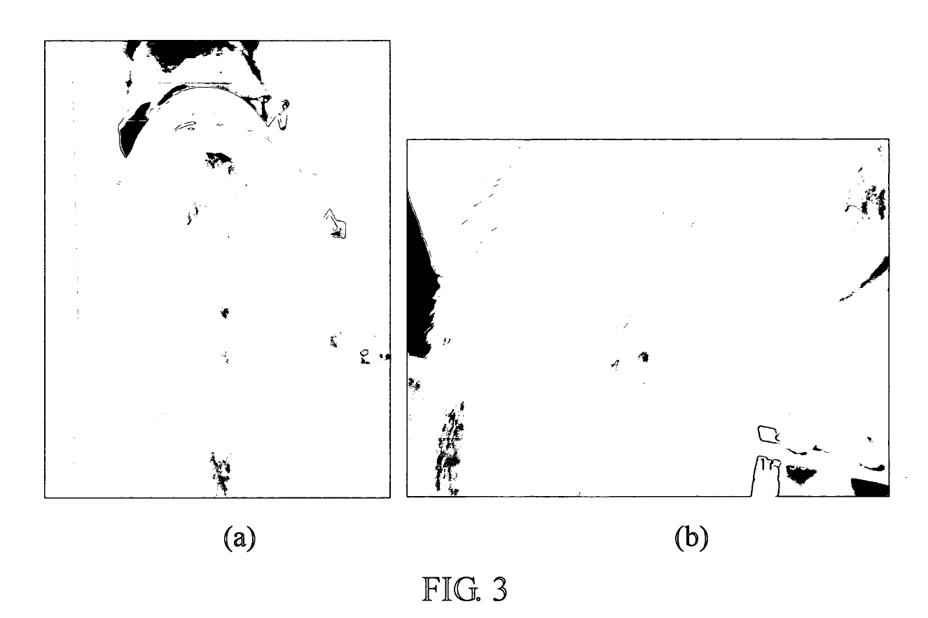

The composition of the present invention was administered for one week after treatment with medical herb itch-relieving agents for one week. The results are shown in FIG. 3. FIG. 3(a) illustrates the subject before administration of the composition of the present invention. FIG. 3(b) illustrates the subject after administration of the composition of the present invention. Obviously, the aforementioned conditions were improved, hair of the dog gradually recovered, and dandruff was relieved after administration of the composition of the present invention. The skin was in a healthy state. So far, the aforementioned conditions do not occur again.

### Example 4

Composition #4 was taken, comprising the components as shown in Table 4.

TABLE 4
Composition #4

| | Plant | Weight (%) |
|---|---|---|
| | Sophorae Radix | 12.9 |
| skin-pathogen-expelling | — | 0.0 |
| plant | | |
| diuresis-inducing plant | — | 0.0 |
| heat-clearing plant | — | 0.0 |
| spleen-invigorating plant | — | 0.0 |
| dampness-removing | | |
| plant | | |
| vital-energy-invigorating | Ginseng Radix | 45.2 |
| plant | | |
| blood-nourishing plant | Angelicae Radix | 41.9 |

Weight (%) means the weight percentage of the extract of the plant.

A 2-month-old female mongrel was employed in this example. Clinically, the conditions of erythema, itchness, and dandruff were observed. Itch-mites were observed by microscopic observation. The dog was diagnosed to suffer from itch-mite infection.

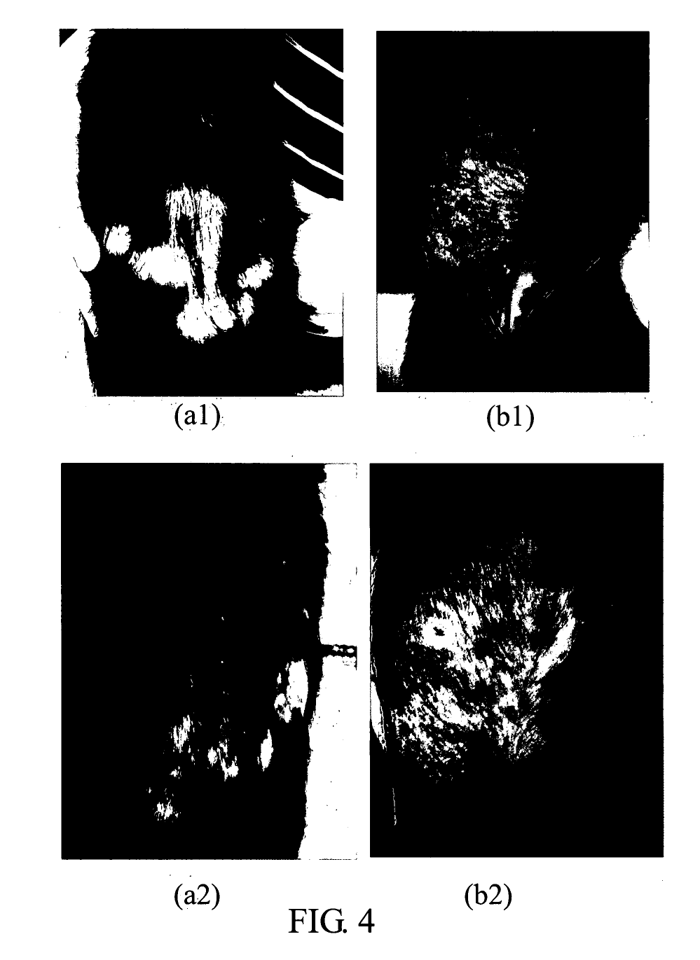

The subject was debugged and injected with anti-histamine, and administered with Ivomac&reg and itch-relieving agents for one week followed by administering the composition of the present invention for two weeks. It was found that the hair of the subject was in a healthy state. The conditions before treatment are shown in FIG. 4(a1), which illustrates the dorsal lesions and FIG. 4(b1) illustrates ventral lesions. The conditions of the subject were obviously relieved after administration of the composition, and hair of the subject was in a healthy state, as shown in FIG. 4(a2) and FIG. 4(b2) which are the dorsal and ventral perspectives, respectively.

### Example 5

Composition #5 was taken, comprising the components as shown in Table 5.

Composition #5

| | Plant | Weight (%) |
|---|---|---|
| | Sophorae Radix | 3.2 |
| skin-pathogen-expelling | — | 17.7 |
| plant | | |
| diuresis-inducing plant | — | 16.1 |
| heat-clearing plant | — | 33.9 |
| spleen-invigorating plant | — | 14.5 |
| dampness-removing | | |
| plant | | |
| vital-energy-invigorating | Ginseng Radix | 6.5 |
| plant | | |
| blood-nourishing plant | Angelicae Radix | 8.1 |

Weight (%) means the weight percentage of the extract of the plant.

A female Mongrel aged 2 months and weighting 5 Kg was employed in this example and the dog was diagnosed to suffer from Alopecia.

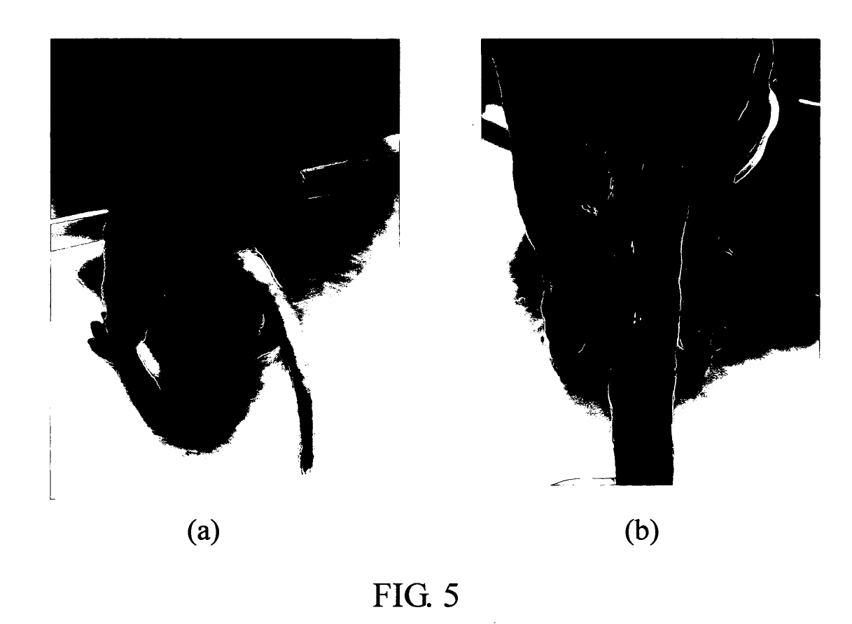

The subject was injected with antibiotics and anti-histamine to relieve itchness, followed by administration of the composition of the present invention for one week. It was found that the hair was in a healthy state. The conditions before administration of the composition are shown in FIG. 5(a). FIG. 5(b) shows that the conditions of the subject were obviously relieved after administration of the composition, and the hair was in a healthy state. The composition was obviously effective for promoting the growth of hair.

Various compositions were tested and the composition # 5 exhibited the best efficacy. Therefore, composition #5 was employed in subsequent tests.

### Example 6

A male Husky aged one year and 9 months and weighing 5 Kg was employed in this example. Clinically, the conditions of a significant amount of ventral erythema, papules, and pustules were observed. Furthermore, the left forelimb of the subject had lesions of erythema, Alopecia and increasing dandruff. The dog was diagnosed to suffer from superficial pyoderma.

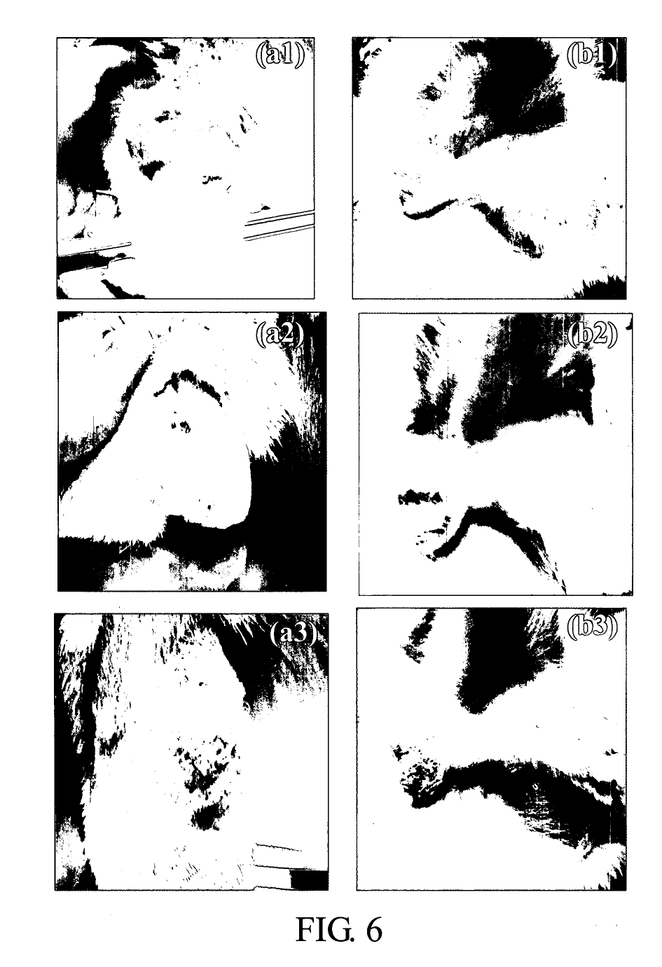

The subject was injected with antibiotics and anti-histamine once, orally administered with itch-relieving agents for 3 days, followed by administration of composition #5 of the present invention for one week. The results are shown in FIG. 6. FIG. 6(a1) shows the lesions of the left forelimb. FIG. 6(b1) shows the ventral lesions. The lesion areas showed substantial erythema or papules before administration of the composition. FIGS. 6 (a2) and (b2) show that the conditions on left forelimb and abdominal part were improved after oral administration of itch-relieving agents for 3 days. FIGS. 6 (a3) and (b3) show that the conditions on left forelimb and abdominal part were relieved much and the hair grew extensively after oral administration of composition #5 for one week.

### Example 7

A female mongrel aged 2 years and 3 months and weighing 3 Kg was employed in this example. Clinically, the conditions of extensive demodex infection (the subject is injected with Ivomac to debug), systemic alopecia, crust, erosion, pruritu, scale, effluent, and loss of skin elasticity and texture were observed. The subject was diagnosed to suffer from deep pyoderma elicited from demodex, in conjunction with alopecia.

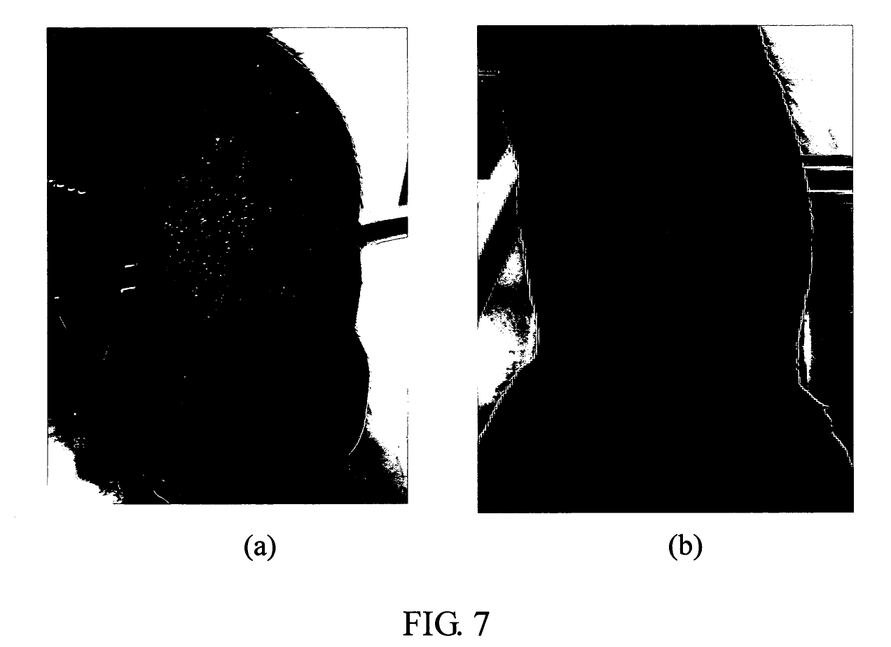

Itch-relieving agents were administered orally for 3 weeks, followed by composition #5 for one week, and the hair grew and recovered to a normal appearance thereafter. FIG. 7(a) shows the conditions before administration, and FIG. 7(b) shows the conditions after administration of the composition. The results demonstrate that the conditions of the subject were relieved and hair grew extensively.

### Example 8

A female Mixed breed cat aged 2 years and 7 months was employed in this example. Clinically, the conditions of shedding at the inner side of the knee joints of the rear legs, and smooth lesions were observed. The cat was diagnosed to suffer from Alopecia.

The hair obviously recovered after oral administration of the composition #5 for one week.

### Example 9

A female Pomeranian aged 10 years dog was employed in this example. Clinically, the hair had never grown for two years after shaving the dog. The aforementioned conditions were possibly due to endocrinal disorder, but no further test was conducted. The dog was diagnosed to suffer from Alopecia.

The hair obviously recovered after oral administration of the composition #5 for one month.

### Example 10

The test was performed on a volunteer, a 38-year-old man (family name—Wen). He was affected by serious shedding during hair-washing (the amount of shed hair is 80-100 each time), and his forehead line showed a clear trend of receding.

After he continuously took composition #5 for one month, it was found that the amount of shed hair decreased to 20-30 each time, and the forehead showed the growth of thin hair and thickening of existing hair. The forehead hairline moved forward after one month, and generally the hair became thick. Aside from clear growth of hair, the skin of the subject also improved, which turned pink and delicate.

### Example 11

The test was performed on a volunteer, a 23-year-old man (family name—Shun). He was affected by serious shedding during hair-washing (the amount of shed hair is 80 each time), and his forehead hairline showed a clear trend of receding while the hair on the top of the head was thinning.

After he continuously took composition #5 for one month, it was found that the amount of shed hair decreased to about 40 each time, existing hair thickened, and generally the hair became thicker, especially the hair on the top of the head. Aside from clear growth of hair, the skin of the subject also improved, which turned pink and delicate.

### Example 12

The test was performed on a volunteer, a 23-year-old man (family name—Li). He was affected by increasing shedding of hair during hair-washing (the amount of shed hair is 100 each time), and his forehead line at both sides showed a clear trend of receding.

After he continuously took composition #5 for one week, it was found that the amount of shed hair decreased to about 40 each time, and some new hair was growing at the forehead.

### Example 13

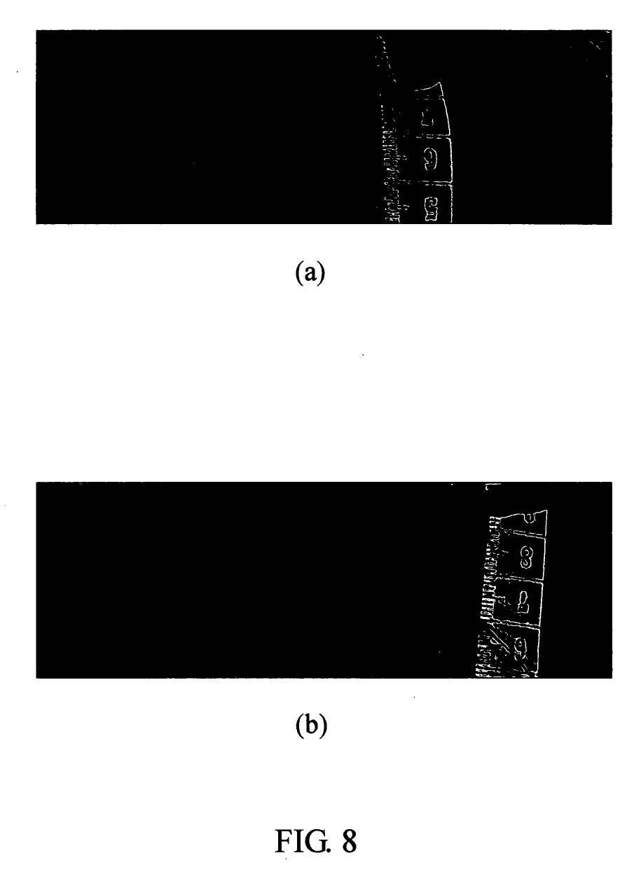

The test was performed on a volunteer, a 45-year-old female (family name—Kao). She was affected by serious shedding during hair-washing (the amount of shed hair is 150 each time), and her forehead line at both sides showed a clear trend of receding. The amount of shed hair decreased to about 100 each time after administration of composition #5 for one week, and further decreased to 80 each time and some new hair was growing at the forehead after administration for 3 weeks. The efficacy of the composition is clearly identified by comparing the photo taken at the 37^{th} day after the first administration of the composition (FIG. 8(b)) to that taken at 5^{th} day (FIG. 8(a)). Hair at the forehead line became thicker (as shown in FIG. 8(b)), and texture of the skin of the subject improved after continuous administration of the composition for one month.

### Example 14

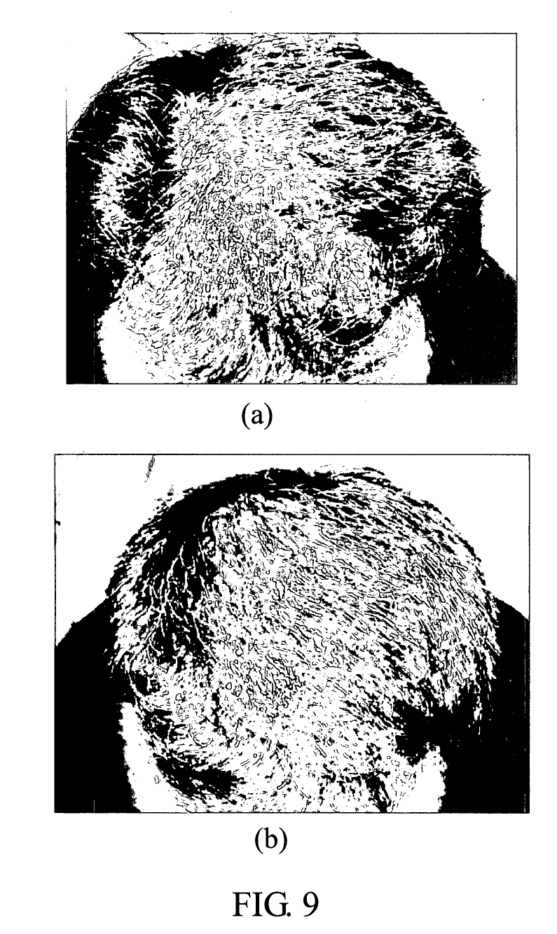

The test was performed on a volunteer, a 29-year-old man (family name—Lin). He was affected by shedding, and his forehead hairline showed a clear trend of receding while the hair on the top of the head was thinning. He was diagnosed to suffer from Male Pattern Baldness. He ever administrated Finasteride, but the effect was poor. After he continuously took composition #5 for 3 weeks, it was found that the amount of shed hair decreased obviously. After he continuously took composition #5 for 3 months, his hair generally became thick. The efficacy of the composition is clearly identified by comparing the photo taken after the administration of the composition for 1 month (FIG. 9(b)) to that taken before the administration of the composition (FIG. 9(a)). His hair at the forehead and the top of the head became thicker. In addition, after continuous administration of the composition for 1 month, his eyesight was 150 degree less than that before the administration of the composition. Thereby, the shedding condition, the texture of the skin, and the eyesight of the subject improved obviously after continuous administration of the composition for 1 month.

Based on the above results, it can be concluded that the medical herb composition of this invention not only relieves conditions of skin disorders, but also promotes the growth of hair and improves skin texture.

According to the examples, it is found that administering an extract of Sophorae Radix in conjunction with an extract of at least one vital-energy-invigorating plant (Ginseng Radix) and an extract of at least one blood-nourishing plant (Angelicae Radix), and contingently an extract of at least one skin-pathogen-expelling plant (e.g. Ledebouriellae Radix/Notopterygii Rhizoma/Cimicifugae Rhizoma), an extract of at least one diuresis-inducing plant (e.g. Alismatis Rhizoma/Polyporus/Atractylodis Rhizoma), an extract of at least one heat-clearing plant (e.g. Puerariae Radix/Scutellariae Radix/Anemarrhenae Rhizoma/Glycyrrhizae Radix/Artemisiae Capillaris Herba), and/or an extract of at least one spleen-invigorating and dampness-removing plant (e.g. Atractylodis Rhizoma) to dogs, cats, and humans can relieve the skin disorder conditions and facilitate the growth of hair.

It has been commonly expected by hair-growth-aid agent researchers that the stage from activation of hair follicles to budding thin hair usually takes generally 3 weeks for dogs and 3 months for men. However, by taking the medical herb composition of the present invention, the significant growth of hair occurs at shedding spots of dogs within one week; and for humans, the amount of shed hair decreases and budding thin hair is clearly visible at the forehead by continuous administering the composition of the present invention for one to two weeks, and robust hair growth is seen by continuous administering the composition of the present invention for one month. Thus, the medical herb composition of the present invention can inhibit skin disorders as well as promote the growth hair, and the efficacy is better than the hair-growth aids known in the art.

The above Examples are presented only for illustrative purpose, and it is not intended that the scope of this invention is limited to these Examples.

What is claimed is:

1. A medical herb composition for promoting the growth of hair of a mammal, comprising:
an extract of Sophorae Radix;
an extract of at least one vital-energy-invigorating plant selected from the group consisting of: Ginseng Radix, Astragali Radix, Batatatis Rhizoma, Zizyphi Fructus, and Tremella; and
an extract of at least one blood-nourishing plant selected from the group consisting of: Angelicae Radix, Rehmanniae Preparata Radix, Longanae Arillus, and Lycii Fructus, wherein the weight percentage of the extract of Sophorae Radix is 2˜38, the weight percentage of the extract of vital-energy-invigorating plant is 2˜60, and the weight percentage of the extract of blood-nourishing plant is 2˜60.

2. The medical herb composition of claim 1, wherein the extract of at least one vital-energy-invigorating plant is an extract of Ginseng Radix or/and an extract of Astragali Radix, and the extract of at least one blood-nourishing plant is an extract of Angelicae Radix.

3. The medical herb composition of claim 1, further comprising: 0.1-50 wt % of an extract of at least one skin-pathogen-expelling plant, 0.1-50 wt % of an extract of at least one diuresis-inducing plant, 0.1-70 wt % of an extract of at least one heat-clearing plant, and/or 0.1-40 wt % of an extract of at least one invigorating and dampness-removing plant.

4. The medical herb composition of claim 1, further comprising: 5-40 wt % of an extract of at least one skin-pathogen-expelling plant, 5-40 wt % of an extract of at least one diuresis-inducing plant, 5-60 wt % of an extract of at least one heat-clearing plant, and/or 2-30 wt % of an extract of at least one spleen invigorating and dampness-removing plant.

5. The medical herb composition of claim 3, wherein the skin-pathogen-expelling plant is selected from the group consisting of: Ledebouriellae Radix, Notopterygii Rhizoma, Cimicifugae Rhizoma, Allii Fistulosi Bulbus, Asari Herba Cum Radice, Ephedrae Herba, Perillae Folium, and Zingiberis Rhizoma;
the diuresis-inducing plant is selected from the group consisting of: Alismatis Rhizoma, Polyporus, Atractylodis Rhizoma, Akebiae Caulis, Aristolochiae Fangchi Radix, and Boehmeriae Radix;
the heat-clearing plant is selected from the group consisting of Puerariae Radix, Scutellariae Radix, Anemarrhenae Rhizoma, Glycyrrhizae Radix, Artemisiae Capillaris Herba, Rehmanniae Radix, and Moutan Radicis Cortex; and
the spleen invigorating and dampness-removing plant is selected from the group consisting of: Atractylodis Rhizoma, Eupatorii Herba, Magnolia Cortex, and Pogostemi Herba.

6. The medical herb composition of claim 5, wherein the extract of at least one skin-pathogen-expelling plant is an extract of Ledebouriellae Radix, an extract of Notopterygii Rhizoma, or/and an extract of Cimicifugae Rhizoma;
the extract of at least one diuresis-inducing plant is an extract of Alismatis Rhizoma, an extract of Polyporus, or/and an extract of Atractylodis Rhizoma;
the extract of at least one heat-clearing plant is an extract of Puerariae Radix, an extract of Scutellariae Radix, an extract of Anemarrhenae Rhizoma, an extract of Glycyrrhizae Radix, or/and an extract of Artemisiae Capillaris Herba; and
the extract of at least one spleen invigorating and dampness-removing plant is an extract of Atractylodis Rhizoma.

7. The medical herb composition of claim 1, wherein the extracts are extracted by water or alcohol.

8. The medical herb composition of claim 1, further comprising an excipient.

9. The medical herb composition of claim 1, wherein the extracts are granulated by spray-drying or freeze-drying.

10. The medical herb composition of claim 1, wherein the medical herb composition is used for inhibiting skin disorders of a mammal simultaneously.

11. The medical herb composition of claim 1, wherein the medical herb composition is an oral formulation.

12. The medical herb composition of claim 1, wherein the medical herb composition is used for improving skin conditions of a mammal simultaneously.

13. A method for preparing a medical herb composition for promoting the growth of hair of a mammal, comprising the steps of:
(a) providing an extract of Sophorae Radix, an extract of at least one vital-energy-invigorating plant, and an extract of at least one blood-nourishing plant by individual extraction and drying, and
(b) mixing the dried extracts to form a mixture, wherein the vital-energy-invigorating plant is selected from the group consisting of: Ginseng Radix, Astragali Radix, Batatatis Rhizoma, Zizyphi Fructus, and Tremella; the blood-nourishing plant is selected from the group consisting of: Angelicae Radix, Rehmanniae Preparata Radix, Longanae Arillus, and Lycii Fructus; the weight percentage of the extract of Sophorae Radix is 2˜38, the weight percentage of the extract of the vital-energy-invigorating plant is 2˜60, and the weight percentage of the extract of the blood-nourishing plant is 2˜60.

14. The method of claim 13, wherein in step (a), the extract of at least one vital-energy-invigorating plant is an extract of Ginseng Radix, and the extract of at least one blood-nourishing plant is an extract of Angelicae Radix.

15. The method of claim 13, wherein step (a) further comprises:
providing 0.1-50 wt % of an extract of at least one skin-pathogen-expelling plant, 0.1-50 wt % of an extract of at least one diuresis-inducing plant, 0.1-70 wt % of an extract of at least one heat-clearing plant, and/or 0.1-40 wt % of an extract of at least one spleen-invigorating plant dampness-removing plant.

16. The method of claim 13, wherein step (a) further comprises:
providing 5˜40 wt % of an extract of at least one skin-pathogen-expelling plant, 5˜40 wt % of an extract of at least one diuresis-inducing plant, 5˜60 wt % of an extract of at least one heat-clearing plant, and/or 2˜30 wt % of an extract of at least one spleen-invigorating and dampness-removing plant.

17. The method of claim 15, wherein the skin-pathogen-expelling plant is selected from the group consisting of: Ledebouriellae Radix, Notopterygii Rhizoma, Cimicifugae Rhizoma, Allii Fistulosi Bulbus, Asari Herba Cum Radice, Ephedrae Herba, Perillae Folium, and Zingiberis Rhizoma;
the diuresis-inducing plant is selected from the group consisting of: Alismatis Rhizoma, Polyporus, Atractylodis Rhizoma, Akebiae Caulis, Aristolochiae Fangchi Radix, and Boehmeriae Radix;
the heat-clearing plant is selected from the group consisting of Puerariae Radix, Scutellariae Radix, Anemarrhenae Rhizoma, Glycyrrhizae Radix, Artemisiae Capillaris Herba, Rehmanniae Radix, and Moutan Radicis Cortex; and
the spleen invigorating and dampness-removing plant is selected from the group consisting of: Atractylodis Rhizoma, Eupatorii Herba, Magnolia Cortex, and Pogostemi Herba.

18. The method of claim 17, wherein the extract of at least one skin-pathogen-expelling plant is an extract of Ledebouriellae Radix, an extract of Notopterygii Rhizoma, or/and an extract of Cimicifugae Rhizoma;
the extract of at least one diuresis-inducing plant is an extract of Alismatis Rhizoma, an extract of Polyporus, or/and an extract of Atractylodis Rhizoma;
the extract of at least one heat-clearing plant is an extract of Puerariae Radix, an extract of Scutellariae Radix, an extract of Anemarrhenae Rhizoma, an extract of Glycyrrhizae Radix extracts, or/and an extract of Artemisiae Capillaris Herba; and
the extract of at least one spleen-invigorating and dampness-removing plant is an extract of Atractylodis Rhizoma.

19. The method of claim 13, wherein step (b) further comprises: providing an excipient, and mixing the excipient and the mixture to form the medical herb composition.

20. The method of claim 13, the medical herb composition is used for inhibiting wet-skin disorder of a mammal simultaneously.

21. The method of claim 13, the medical herb composition is an oral formulation.

22. The method of claim 13, wherein the extracts are extracted by water or alcohol.

23. The method of claim 13, wherein the medical herb composition is used for improving skin conditions of a mammal simultaneously.
